(19) United States
(12) Patent Application Publication
(10) Pub. No.: US 2003/0157670 A1
(43) Pub. Date: Aug. 21, 2003

(54) Capsaicin-decomposing/synthesizing enzymes and a method for producing the same

(75) Inventors: Kazuhiro Nakanishi, Okayama City (JP); Takaharu Sakiyama, Okayama City (JP); Koreyoshi Imamura, Okayama City (JP)

Correspondence Address: OLIFF & BERRIDGE, PLC, P.O. BOX 19928, ALEXANDRIA, VA, 22320, US

(73) Assignee: OKAYAMA UNIVERSITY, Okayama City (JP)

(21) Appl. No.: 10/196,280

(22) Filed: Jul. 17, 2002

(30) Foreign Application Priority Data
Jan. 17, 2002 (JP) 2002-8, 934

(51) Int. Cl. C12P 13/02; C12N 9/80; C07H 21/04; C12P 21/02; C12N 1/21; C12N 15/74

(52) U.S. Cl. 435/129; 435/69.1; 435/252.3; 435/320.1; 435/228; 536/23.2

(57) ABSTRACT

Capsaicin decomposing/synthesizing enzymes are disclosed, which each have the following physical and chemical properties: (1) function and substrate specificity: the enzyme catalyzes a decomposition and/or synthesis reaction of capsaicin and/or capsaicin analogs; (2) an optimal temperature range: near 55° C.; and(3) an optimal pH range of 7-8. Microorganisms to be used for producing these enzymes are also disclosed.

### BACKGROUND OF THE INVENTION

(1) Field of the Invention

The present invention relates to novel enzymes, microorganisms being capable of producing said enzymes, methods for preparing said enzymes, and a method for synthesizing useful materials by utilizing said enzymes. More particularly, the invention relates to novel enzymes for catalyzing decomposition and/or synthesis of capsaicin and/or capsaicin analogs; microorganisms being capable of produce said enzymes, methods for preparing said enzymes, and a method for synthesizing capsaicin or capsaicin analogs by utilizing said enzymes.

Capsaicin can be not only naturally used as a pungent food ingredient in seasonings, but also used as healthy/diet foods and supplement ingredient because capsaicin activates body metabolism. In addition, capsaicin has a pain-relieving function and anti-itching function as a sensor neuron-interrupting agent to selectively interrupt nociceptor and sensor receptor on topical application, subcutaneous administration or the like. Furthermore, capsaicin has a skin temperature-increasing function, a stamachic function, etc. Therefore, capsaicin can be used ingredients for various medicines. Since the capsaicin analogs are expected to have effects similar to those of capsaicin, it is considered that the analogs can be used as ingredient for healthy/diet foods, supplements, medicines, etc.

The decomposition of capsaicin and the capsaicin analogs is useful in that amine components as decomposed products are starting materials enzymatically synthesizing the capsaicin analogs. That is, various capsaicin derivatives can be synthesized by binding fatty acids different from original ones to the amine components obtained by the decomposition with enzymes of the capsaicin and the capsaicin analogs, by means of those enzymes.

(2) Related Art Statement

Capsaicin is a kind of main pungent ingredients of Capsicum annuum, and has a useful physiological activities such as appetite improvement action and analgesic action. Because capsaicin has various physiological activities like this, it is useful in technical fields of functional food materials and raw materials for medicines. Thus, capsaicin has now been attracting attention over the world.

Capsaicin and capsaicin analogs can be synthesized by organic synthetics methods, for example.

As a synthesis method utilizing an enzyme, it is known that capsaicin analogs were enzymatically synthesized with success by exerting a lipase upon vanillylamine and triglyceride.

Further, it is also known that capsaicin analogs can be synthesized by using a rat lever acetone powder.

However, the above organic synthetic methods have a defect that since reagents used are not allowed for processing foods, the methods cannot be used for producing food ingredients. Furthermore, both the enzyme-utilizing syntheses method and the rat lever acetone powder-utilizing method as mentioned above have unfavorably low synthesis yields, the former having a yield of not more than 20%, and the latter having a yield of 8 to 28% even in two days. In addition, mass production is difficult for industrial applications in the latter method because the enzyme-catalyzed products are obtained by using rat levers as a starting material. Therefore, a method for the preparation of capsaicin and its analogs, in which they can be easily and stably mass produced at a high yield, has been desired.

### SUMMARY OF THE INVENTION

It is an object to provide enzymes which are useful in easily and stably mass producing capsaicin or capsaicin analogs at a high yield.

It is another object of the present invention to provide methods for preparing said enzymes.

It is a further object of the present invention to provide microorganisms being capable of producing said enzymes.

It is a still further object of the present invention to provide a method for synthesizing capsaicin and/or capsaicin analogs by utilizing said enzymes.

In order to accomplish the above objects, the present inventors have made investigations on various microorganisms, looking for enzymes having excellent ability to specifically hydrolyze and/or synthesize capsaicin and capsaicin analogs. As a result, the present inventors discovered the enzymes and the production methods of these enzymes according to the present invention.

The capsaicin decomposing/synthesizing enzyme according to the present invention has the following physical and chemical properties (1) to (3):

(1) function and substrate specificity: the enzyme catalyzes a decomposition and/or synthesis reaction of capsaicin and/or capsaicin analogs;

(2) an optimal temperature range near 55° C.; and

(3) an optimal pH range of 7-8.

The capsaicin decomposing/synthesizing enzyme is an enzyme originating from a microorganism belonging to a Streptomyces genus as a preferred embodiment.

The microorganism belonging to the Streptomyces genus according to the present invention is a Streptomyces mobaraensis IFO 13819 or a Streptomyces luteoreticuli IFO 13422 in a preferred embodiment thereof.

The microorganism according to the present invention is a microorganism belonging to a Streptomyces genus and being capable of producing a capsaicin and/or capsaicin analogs having the following physical and chemical properties:

(1) function and substrate specificity: the enzyme catalyzes decomposition and/or synthesis reaction of capsaicin and/or capsaicin analogs;

(2) an optimal temperature range: near 55° C; and

(3) an optimal pH range of 7-8.

The method for producing a capsaicin decomposing/synthesizing enzyme according to the present invention comprises the steps of incubating a microorganism belonging to the Streptomyces genus and being capable of producing the capsaicin decomposing/synthesizing enzyme, and collecting the capsaicin decomposing/synthesizing enzyme from a culture thereof.

The microorganism is Streptomyces mobaraensis IFO 13819 or Streptomyces luteoreticuli IFO 13422 in a preferred embodiment of the method for producing the capsaicin decomposing/synthesizing enzyme according to the present invention.

The method for synthesizing capsaicin or a capsaicin analog according to the present invention comprises the step of reacting vanillylamine with a fatty acid in the presence of the enzyme as mentioned above.

These and other objects, features and advantages of the invention will be appreciated upon reading of the following description of the invention when taken in conjunction with the attached drawings, with the understanding that some modifications, variations and changes of the same could be easily made by the skilled person in the art to which the invention pertains.

In a preferred embodiment of the method for synthesizing capsaicin or a capsaicin analog according to the present invention, the above reaction is performed in the presence of cobalt ions.

### BRIEF DESCRIPTION OF THE DRAWINGS

For a better understanding of the invention, reference is made to the attached drawings, wherein:

FIG. 1 is a graph showing results of a purified enzyme, SDS-PAGE as one embodiment of the present invention.

FIG. 2 is a graph showing the pH dependency of the enzyme upon the activity.

FIG. 3 is a graph showing the heat stability of the purified enzyme as a further embodiment of the present invention.

FIG. 4 is a graph showing results in hydroxyapatite column chromatography;

FIGS. 5(a) and 5(b) are graphs showing results in hydroxyapatite column chromatography, FIGS. 5(a) and 5(b) being first and second hydroxyapatite column chromatography results, respectively.

FIG. 6 is a graph showing the dependency of enzymatic activity of the purified enzyme as the embodiment of the invention upon the temperature.

FIG. 7 is a graph showing the history of a synthesis reaction of a capsaicin derivative.

The capsaicin decomposing/synthesizing enzyme according to the present invention widely means enzymes that catalyze the hydrolysis and/or the synthesis of capsaicin and/or the capsaicin analogs. More specifically, the capsaicin decomposing/synthesizing enzyme according to the present invention has the following physical and chemical properties: (1) function and substrate specificity—the enzyme catalyzes a decomposition and/or synthesis reaction of capsaicin and/or capsaicin analogs; (2) an optimal temperature range: near 55° C.; and (3) an optimal pH range of 7-8. The decomposing/synthesizing enzyme according to the present invention has the molecular weight in a range of 45 kDa to 60 kDa. The molecular weight of the enzyme according to the present invention is determined to be 60 kDa by SDS-PAGE method, whereas the molecular weight of the same enzyme is determined to be around 45 kDa by using HPLC gel chromatography. Since the enzyme exhibits a single band in SDS-PAGE, the enzyme is considered a monomer.

The capsaicin decomposing/synthesizing enzyme according to the present invention is preferably an enzyme originated from the Streptomyces genus. The enzyme according to the present invention is preferably originated from Streptomyces mobaraensis IFO 13819 or Streptomyces luteoreticuli IFO 13422 among the microorganisms belonging to the Streptomyces genus.

The enzymatic activity of the capsaicin decomposing/synthesizing enzyme according to the present invention is promoted preferably in the presence of cobalt ions. The enzyme having the enzymatic activity as promoted like this is used to advantageously synthesize capsaicin or capsaicin analog at a high efficiency as mentioned later.

In the method for synthesizing capsaicin or the capsaicin analog according to the present invention, the decomposing/synthesizing enzyme according to the present invention as mentioned above is used. More specifically, vanillylamine is reacted with a fatty acid in the presence of the above decomposing/synthesizing enzyme. The reaction formula is shown below.

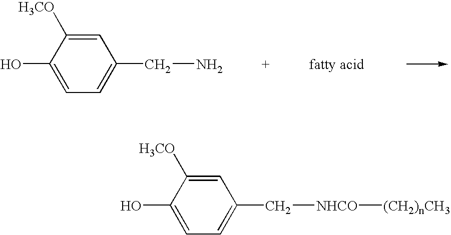

Similarly, the fatty acid is not particularly limited, and various fatty acids may be used corresponding to the desired capsaicin analogs. For example, octanoic acid, decanoic acid, lauric acid, myristic acid, palmitic acid, stearic acid. etc. may be used.

The method for synthesizing capsaicin or capsaicin analog according to the present invention can be easily applied to a case where an analogous amine similar to vanillylamine is used to obtain a desired capsaicin analog through reacting them with the fatty acid. Such a case will fall in the scope of the synthesizing method of the present invention.

If a fatty acid having the number of carbons greater than that of palmitic acid is used, a reactive product may not be dissolved in some solvent. Therefore, the reaction may be carried out in a solvent appropriately selected. In this case, it may be that the amount of the solvent is increased or cobalt ions are added to promote the reaction.

The microorganism according to the present invention is a microorganism belonging to a Streptomyces genus and being capable of producing a capsaicin decomposing/synthesizing enzyme having the following physical and chemical properties:

(1) function and substrate specificity: the enzyme catalyzes decomposition and/or synthesis reaction of capsaicin and/or capsaicin analogs;

(2) an optimal temperature range: near 55° C.; and

(3) an optimal pH range of 7-8.

As mycological properties of the microorganisms belonging to the Streptomyces genus, it may be recited that the microorganism belongs to actinomycetes, and is Gram-positive obligate aerobe growing in a branched filamentous form.

Considering that the microorganism should have a high productivity of the capsaicin decomposing/synthesizing enzyme according to the present invention, Streptomyces mobaraensis IFO 13819 or Streptomyces luteoreticuli IFO 13422 is preferably used as the microorganism.

Streptomyces mobaraensis IFO 13819 and Streptomyces luteoreticuli IFO 13422 are available from Institute for Fermentation, Osaka (IFO) (now transferred to “National Institute of Technology and Education Biological Resource Center”, NBRC, with resect to the general microorganism strain assigning business) in Japan, and IFO 13919 and IFO 13422 are the numbers of deposits.

The same strain as IFO13819 has been deposited in other culture collection institutes of microoganisms. The numbers of deposits are JCM4168 in the Japan Collection of Microorganisms (JCM) of RIKEN (The Institute of Physical and Chemical Research), ATCC29032 in American Type Culture Collection (ATCC) in U.S.A., and NRRL B-3729 in National Center for Agricultural Utilization Research (NRRL) in U.S.A. The same strain as IFO 13422 has been deposited as Streptomyces mobaraensis ATCC27446 in ATCC.

The capsaicin decomposing/synthesizing enzyme according to the present invention can be produced by incubating a microorganism belonging to the Streptomyces genus and being capable of producing this enzyme in an ordinary way, and collecting said enzyme from the culture thereof. The decomposing/synthesizing enzyme according to the present invention can be collected from an incubated product of a spontaneous or artificial variant of the above microorganism. As a mode of the incubation, liquid incubating, solid incubating, etc. may be recited. Any of them is available for the invention. In order to industrially advantageously incubate enzyme, shake incubating, ventilation stirring incubating, etc. may be employed.

As sources of nutrients are not particularly limited. A carbon source, a nitrogen source, etc. which are ordinarily used for incubating the microorganisms may be recited. As the carbon source, a yeast extract, glycerin, glucose, etc. may be used. As the nitrogen source, organic nitrogen compounds such as peptone, meat extract and corn steep liquor may be recited. In addition, one or more inorganic salts including sodium chloride, phosphate salts, sulfate salts and other metal salts of potassium, magnesium, calcium and zinc may be added into the culture, for example, if appropriate. The incubating conditions such as incubating temperature and time may be appropriately selected as being suitable for the growth of the microorganism used and affording the maximum production of the decomposing/synthesizing enzyme according to the present invention. For example, pH of the culture is near neutral, preferably 6.0-8.0, more preferably around 7.0. The growing temperature of the actinomycetes is generally 28˜37° C. The incubating temperature for their microorganism bodies is preferably in a range of 28˜32° C. Particularly, it is recommended that the incubating temperature for two strains:Streptomyces mobaraensis IFO 1389 and Streptomyces luteoreticuli IFO 13422 as mentioned later is 28° C.

The capsaicin decomposing/synthesizing enzyme according to the present invention may be collected from the cultured mixture thus produced can be obtained by using an appropriate method commonly used for collecting metabolic products. For example, as such a method, a method utilizing a difference in solubility between the decomposing/synthesizing enzyme and impurities, a method utilizing affinity, a method utilizing difference in molecular weight and/or the like may be used singly or in combination or repeatedly. For example, since the decomposing/synthesizing enzyme according to the present invention is secreted outside the microorganism, the enzyme may be obtained in a purified state by the steps of incubating the microorganism, obtaining a culture supernatant liquid through removal of the microorganism from the culture via filtration or centrifugal separation, and subjecting the supernatant to salting-out with ammonium sulfate, various ion exchange chromatographies, gel filtration chromatographies, etc. in combination.

These and other objects, features and advantages of the invention will be apparent from the following description of the invention when taken in conjunction with the attached drawings, with the understanding that some modifications, variations and changes could be easily made by the skilled person in the art.

### EXAMPLES

The present invention will be explained in more detail with reference to the following examples, but the invention should never be interpreted as being limited to them.

### Example 1

(Purification of Capsaicin Decomposing/Synthesizing Enzymes)

First, whether a capsaicin decomposing/synthesizing enzyme was contained in a greater amount inside or outside microorganism bodies was examined. More specifically, by using Streptomyces mobaraensis IFO 13819 as screened effective, activity was measured with respect to a solution obtained by ultrasonically crushing the microorganism bodies and a culture supernatant, which revealed that the specific activity outside the microorganism bodies increased with the lapse of incubating days, whereas that inside the microorganism bodies almost disappeared in a incubating period of 6 days. Therefore, it is considered that the capsaicin decomposing/synthesizing enzyme according to the present invention as produced by the microorganism bodies is effectively secreted outside the microorganism bodies.

Various Streptomyces species were subjected to screening so as to select those having high capsaicin decomposing/synthesizing enzymatic activity. A screening method is as follows.

A culture was prepared with use of 4% beef extract, 2% polypepton, 2% soluble starch, 0.2% yeast extract, 0.2% of K₂HPO₄ and 0.1% MgSO₄, and incubating was effected at a temperature of 30° C. and an initial pH 7.0 for 7 days under a shakning speed of 120 strokes/min. The activity was measured by HPLC after incubation with 0.13 mM capsaicin as a substrate at 37° C. The enzymatic activity 1U was defined as an amount of the enzyme required for hydrolyzing 1 μmol of capsaicin at 37° C. for one hour. Screening results are shown in the following Table. As a result, Streptomyces mobaraensis IFO 13819 and Streptomyces luteoreticuli IFO 13422 showed relatively high enzymatic activities of 1.2 U/mL and 1.0 U/mL, respectively. According to the ATCC classification, Streptomyces luteoreticuli is classified into Streptomyces mobaraensis.

| | IFO NO. | Activity (UmL) |
|---|---|---|
| S. mobaraensis | 13819 | 1.2 |
| S. ardus | 13430 | — |
| S. blastmyceticus | 12747 | — |
| S. cacaoi | 13813 | — |
| S. caespitosus | 13490 | — |
| S. cinnamoneus | 12852 | — |
| S. exfolatus | 12319 | — |
| S. griseinus | 12869 | — |
| S. lividoclavatus | 13870 | — |
| S. lividus | 13787 | — |
| *S. luteoreticuli | 13422 | 1.0 |
| S. mobaraenis | 13476 | — |
| S. olivaceus | 12805 | — |
| S. roseoverticillatus | 12817 | — |
| S. scabiei | 13767 | — |
| S. sioyaensis | 12820 | — |
| S. spheroides | 12917 | — |
| S. toyocaensis | 12824 | — |
| S. tuirus | 15617 | — |
| S. venezuelae | 13097 | — |
| S. violaceoruber | 13385 | — |

Based on the above knowledge, incubation was effected by using Streptomyces mobaraensis IFO 13819 as strain. A suspension of a sporule of Streptomyces mobaraensis IFO 13819 was inoculated into a liquid culture at pH 7 containing 2.0% of soluble starch, 2.0% of polypepton, 4.0% of meat extract, 0.2% of yeast extract and 0.2% of potassium hydrogen phosphate and 0.1% of magnesium sulfate, followed by incubation at 30° C. for 7 days.

Then, ammonium sulfate was added to a culture so that supernatant might be saturated at 50% with ammonium sulfate, thereby precipitating the enzyme with ammonium sulfate. The resulting precipitate was dissolved into a buffer: 50 mM NaCl/25 mM Tris-HCl (pH 7.2), fractionated with CM Sephadex C-50, and purified twice with hydroxyapatite column chromatography.

In the following, a flow chart of the purification of the enzyme is shown below.

Culture Filtrate (600 mL)

Ammonium sulfate (final concentration=50%)

Precipitate

50 mM NaCl/25 mM Tris-HCl (pH 7.2) dialysis at 4° C. (3L×3 times)

Active Fraction (70 mL)

CM-Sephadex C50 column (1.6 i.d.×35 cm) elution buffer; linear increase of NaCl concentration in the 25 mM Tris-HCl buffer from 50 to 500 mM flow rate; 0.35 mL/min

Active Fraction

Hydroxqapatite column (1.6 i.d.×18 cm) elution buffer; linear increase of potassium phosphate buffer concentration from 40 to 400 mM (pH 7.2) flow rate; 0.18 mL/min

Active Fraction

Hydroxyapatite column (1.6 i.d.×18 cm) elution buffer; linear increase of the potassium phosphate concentration from 40 to 400 mM flow rate; 0.15 mL/min

Active Fraction

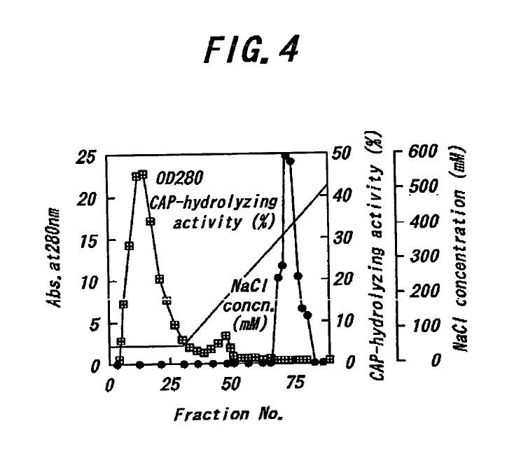

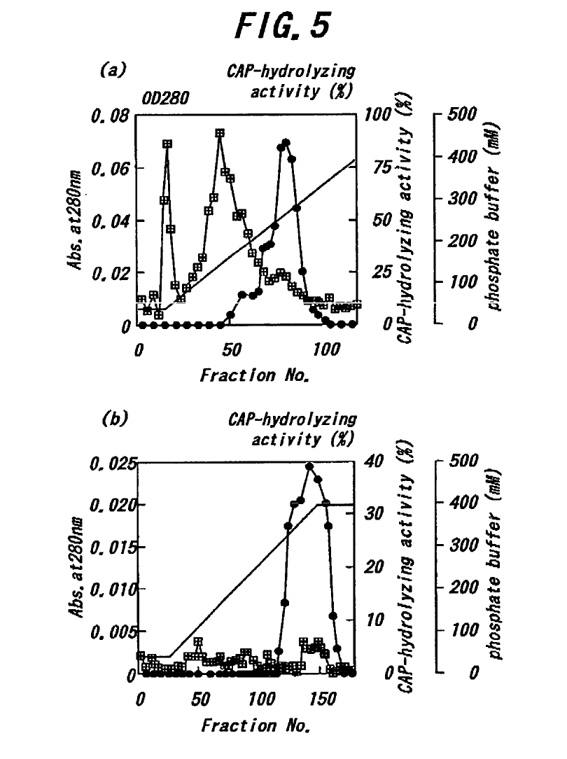

FIG. 4 shows an fluting curve obtained by the CM-Sephadex C-50 column chromatography. The concentration of proteins and the hydrolysis activity of capsaicin (CAP-hydrolysis activity) were represented by OD280 and the hydrolysis percentage, respectively, in FIG. 4. It is seen from FIG. 4 that the active fraction was eluted around an NaCl concentration of 400 mM. This active fraction was further fractionated with the hydroxyapatite chromatography (FIG. 5(a)). Since impurities were recognized mixed in this active fraction, it was fractioned again with the hydroxyapatite chromatography. Results are shown in FIG. 4(b), which shows that the enzyme was obtained in a pure form at near a phosphoric acid concentration of 350 mM.

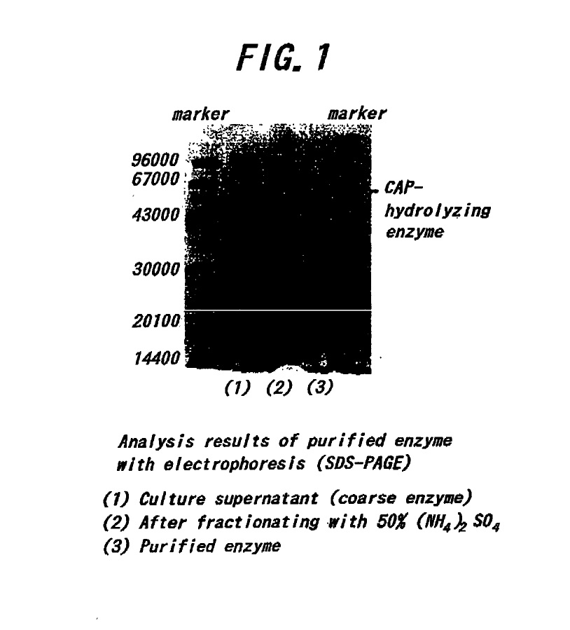

In this way, a supernatant containing the enzyme was collected by centrifugally removing the microorganism. Next, the activity of the supernatant was examined, which revealed that the entire activity of the enzyme in 0.6 L of the supernatant was 345 U, and a specific activity was 0.061 U/mg. As mentioned above, the enzymatic activity 1 U was defined as an amount of the enzyme required to hydrolyze 1 gm of capsaicin at 37° C. for one hour. Ammonium sulfate was added into the supernatant so that the supernatant might be saturated at 50% with ammonium sulfate, thereby obtaining a precipitated fraction. This fraction was subjected to cation exchange chromatography (CM-Sephadex) once and hydroxyapatite chromatography twice, so that the enzyme having a specific activity of 197.0 U/mg (Table 1) and purified as a single enzyme (molecular weight of about 60 kDa) by electrophoresis with polyacryl amide gel. See FIG. 1.

TABLE 1

| | | Whole | | Specific | |
|---|---|---|---|---|---|
| | Protein | activities | Yield | activity | Purified |
| | (mg) | (*units) | (%) | (U/mg) | (times) |
| Filtrate of culture | 7475 | 345 | 100 | 0.061 | 1 |
| Ammonium sulfate | 784 | 165 | 47.8 | 0.21 | 3.4 |
| CM-SephadexC-50 | 4.8 | 62.5 | 18.1 | 13.1 | 215 |
| Hydroxyapatite (1) | 0.26 | 37.5 | 10.9 | 143.7 | 2356 |
| Hydroxyapatite (2) | 0.08 | 15.8 | 4.6 | 197.0 | 3230 |

1 Unit: amount of oxygen required to hydrolyze 1 μmol of capsaicin at 37° C. in 1 hour.

The above specific activities were very high as compared with those of various enzymes reportedly having the capsaicin decomposing activity.

Next, the reactivity of the purified enzyme was examined. Examination of the stability of the enzyme against various reagents revealed that the capsaicin hydrolysis activity of the purified enzyme was enhanced by cobalt ions (Table 2). Further, it was shown that the activity was inhibited by addition of PMSF (phenylmethane sulfonylfluoride) known as an inhibitor against serine protease.

TABLE 2
Effects of various reagents upon oxygen activity

| | Concentration (mM) | Relative activity (%) |
|---|---|---|
| Control | 1 | 100 |
| PCMB | 1 | 96.6 |
| Idoacetoamide | 10 | 86.5 |
| β-mercaptoethanol | 1 | 79.3 |
| DTT | 1 | 79.8 |
| MgSO₄ | 1 | 96.0 |
| FeSO₄ | 1 | 88.8 |
| CaCl₂ | 1 | 101.6 |
| AgNO₃ | 1 | 100.1 |
| ZnCl₂ | 1 | 95.5 |
| CuSO₄ | 1 | 96.9 |
| EDTA | 1 | 85.5 |
| GSH | 1 | 99.8 |
| CoCl₂ | 1 | 145.5 |
| L-Cys | 1 | 101.2 |
| (NH₄) ₆Mo₇O₂₄ | 1 | 88.7 |
| PMSF | 1 | 42.7 |

reaction solution: 0.13 mM capsaicin/50 mM Tris-HCl (pH 7.5)

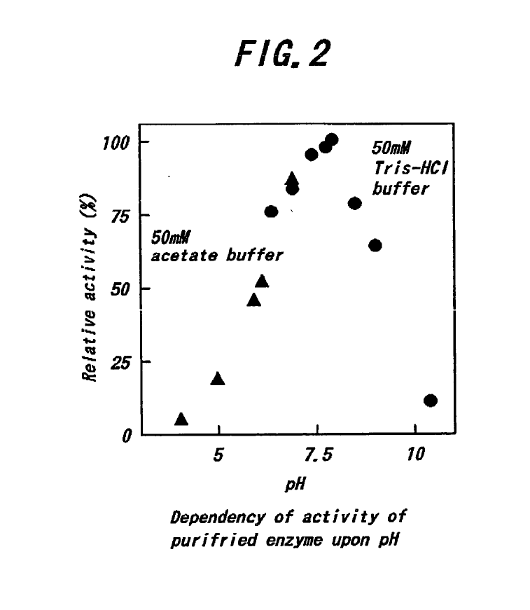

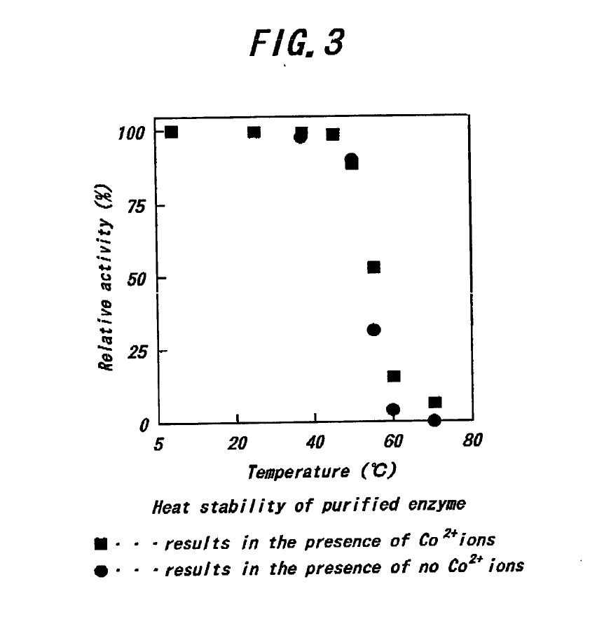

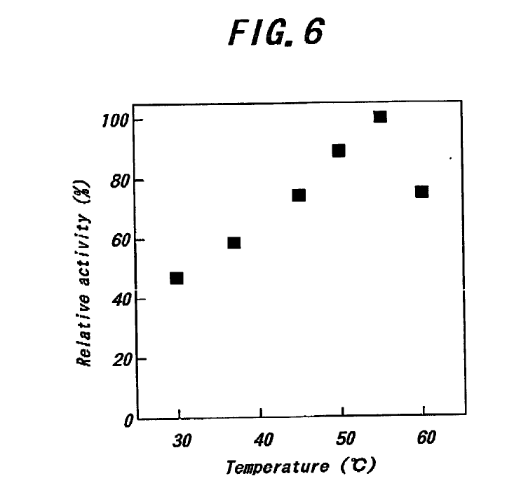

The optimal pH of the capsaicin decomposing/synthesizing enzyme was around pH 7-8 (See FIG. 2). Residual activity was examined after incubation at various temperatures for one hour with respect to a case where cobalt ions were added and a case where no cobalt ions were added. Results are shown in FIG. 3. The enzyme showed such heat resistance that it was stable up to near 50° C. It was also showed that the cobalt ion-added case exhibited higher residual activity at not less than 50° C. (FIG. 3). From these results, it is suggested that the cobalt ions can contribute to promotion of the activity and the stability of the present enzyme. Further, examination of the dependency of the activity of the enzyme upon the temperature was examined, which revealed the highest reactivity at 55° C. (FIG. 6).

### Example 2

Next, capsaicin analogs were synthesized by using the capsaicin decomposing/synthesizing enzyme (purified enzyme from Streptomyces mobaraensis IFO13819) according to the present invention.

Examples showing that the enzyme has the ability to synthesize the capsaicin analogs are as follows. Vanillylamine hydrochloride, 10 mM (final concentration) and lauric acid, 100 mM (final concentration) were reacted with use of 30 U of the enzyme in a dual phase system of water/n-hexane (hexane phase volume/aqueous phase volume=19) for 8 days under stirring, provided that the total reaction liquid amount was 20 ml and the initial pH of the aqueous phase (100 mM tris-hydrochloric acid buffer solution containing 0.5 mM of CoCl₂) was pH 7.3. The reaction temperature was 37° C.

As a comparative example, aminoacylase was used. The resulting capsaicin analogs were quantitatively measured by the HPLC analysis of the hexane phases. Reaction results are shown in Table 3 for the cases where each of the enzymes was used together with various fatty acids.

TABLE 3

| | | Synthesis of capsaicin analogs (mM) | |
|---|---|---|---|
| | Number | | Capsaicin decomposing |
| | of | | synthesizing enzyme |
| Fatty acid | carbons | Aminocylase | of the invention |
| Octanoic acid | C8 | 0.74 | 0.30 |
| Decanoic acid | C10 | 0.61 | 0.43 |
| Lauric acid | C12 | 0.051 | 1.25 |
| Myristic acid | C14 | — | 0.24 |
| Palmitic acid | C16 | — | 0.01 |

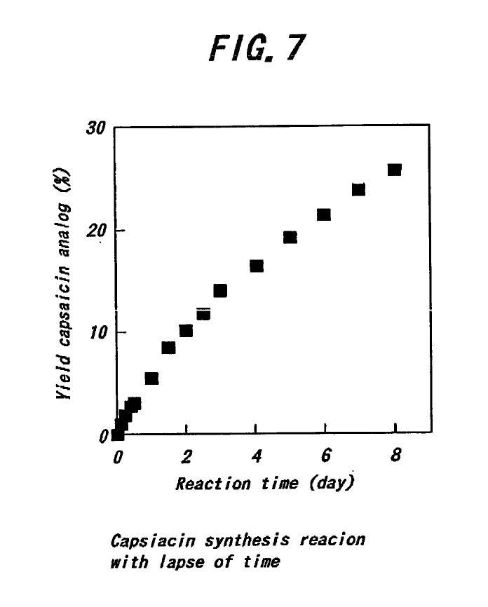

As results, it could be confirmed that the capsaicin analog was synthesized at a high yield (lauric acid, yield: about 28%) (FIG. 7). Further, the enzyme according to the present invention showed that the synthesis reactivity in the cases of vanillylamine and various saturated fatty acids as substrates was highest in lauric acid (C12), and the enzyme acted upon fatty acids having relatively long carbon chains (palmitic acid: C16).

The present invention has been explained in detail based on the embodiments of the present invention with referring to specific examples. However, the present invention is not intended to be limited to the above only, but it is clear to the skilled person in the art to which the invention pertains that various modifications, changes and variations of the invention could be easily made without departing from the gist of the invention.

The present invention advantageously provides the enzymes which are useful in easily and stably mass producing and synthesizing capsaicin and the capsaicin analogs as well as the production method of capsaicin and the capsaicin analogs with use of the above enzymes.

What is claimed is:

1. A capsaicin decomposing/synthesizing enzyme having the following physical and chemical properties (1) to (3):
(1) function and substrate specificity: the enzyme catalyzes a decomposition and/or synthesis reaction of capsaicin and/or capsaicin analogs;
(2) an optimal temperature range: near 55° C.; and
(3) an optimal pH range of 7-8.

2. The capsaicin decomposing/synthesizing enzyme set forth in claim 1, which has a molecular weight of about 60 kDa.

3. The capsaicin decomposing/synthesizing enzyme according to claim 1, which is an enzyme originating from a microorganism belonging to a Streptomyces genus.

4. The capsaicin decomposing/synthesizing enzyme according to claim 2, which is an enzyme originating from a microorganism belonging to a Streptomyces genus.

5. The capsaicin decomposing/synthesizing enzyme according to claim 3, wherein the microorganism belonging to the Streptomyces genus is a Streptomyces mobaraensis IFO 13819 or a Streptomyces luteoreticuli IFO 13422.

6. The capsaicin decomposing/synthesizing enzyme according to claim 4, wherein the microorganism belonging to the Streptomyces genus is a Streptomyces mobaraensis IFO 13819 or a Streptomyces luteoreticuli IFO 13422.

7. A method for producing a capsaicin decomposing/synthesizing enzyme comprises the steps of incubating a microorganism belonging to the Streptomyces genus and being capable of producing the capsaicin decomposing/synthesizing enzyme, and collecting the capsaicin decomposing/synthesizing enzyme from a culture thereof.

8. The method set forth in claim 7, wherein the microorganism is a Streptomyces mobaraensis IFO 13819 or a Streptomyces luteoreticuli IFO 13422.

9. A microorganism, which belong to a Streptomyces genus and is capable of producing a capsaicin and/or capsaicin analogs having the following physical and chemical properties:
(1) function and substrate specificity: the enzyme catalyzes decomposition and/or synthesis reaction of capsaicin and/or capsaicin analogs;
(2) an optimal temperature range: near 55° C.; and
(3) an optimal pH range of 7-8.

10. The microorganism set forth in claim 9, which is Streptomyces mobaraensis IFO 13819 or Streptomyces luteoreticuli IFO 13422.

11. A method for synthesizing capsaicin or a capsaicin analog comprises the step of reacting vanillylamine with a fatty acid in the presence of an enzyme having the following physical and chemical properties (1) to (3):
(1) function and substrate specificity: the enzyme catalyzes a decomposition and/or synthesis reaction of capsaicin and/or capsaicin analogs;
(2) an optimal temperature range: near 55° C.; and
(3) an optimal pH range of 7-8.

12. The method set forth in claim 11, wherein the capsaicin decomposing/synthesizing enzyme has a molecular weight of about 60 kDa.

13. The method in claim 11, wherein the capsaicin decomposing/synthesizing enzyme is an enzyme originating from a microorganism belonging to a Streptomyces genus.

14. The method in claim 12, wherein the capsaicin decomposing/synthesizing enzyme is an enzyme originating from a microorganism belonging to a Streptomyces genus.

15. The method set forth in claim 13, wherein the microorganism belonging to the Streptomyces genus is a Streptomyces mobaraensis IFO 13819 or a Streptomyces luteoreticuli IFO 13422.

16. The method set forth in claim 14, wherein the microorganism belonging to the Streptomyces genus is a Streptomyces mobaraensis IFO 13819 or a Streptomyces luteoreticuli IFO 13422.

17. The method set forth in claim 7, wherein the reaction is performed in the presence of cobalt ions.
